(19) United States
(12) Patent Application Publication
(10) Pub. No.: US 2024/0415768 A1
(43) Pub. Date: Dec. 19, 2024

(54) SUSTAINED-RELEASE GEL WITH INSOLUBLE SALT AS PH ADJUSTER, PREPARATION METHOD AND APPLICATION THEREOF

(71) Applicant: FUDAN UNIVERSITY, SHANGHAI (CN)

(72) Inventors: JIANDONG DING, SHANGHAI (CN); WEN GUO, SHANGHAI (CN); DINGLINGGE CAO, SHANGHAI (CN); LIN YU, SHANGHAI (CN)

(21) Appl. No.: 18/817,861

(22) Filed: Aug. 28, 2024

Related U.S. Application Data
(63) Continuation of application No. PCT/CN2023/096530, filed on May 26, 2023.

(30) Foreign Application Priority Data
Jun. 7, 2022 (CN) 202210638893.7

(51) Int. Cl. A61K 9/06; A61K 31/131; A61K 31/167; A61K 31/198; A61K 31/245; A61K 31/445; A61K 31/455; A61K 31/4745; A61K 31/475; A61K 31/704; A61K 31/7068; A61K 47/34

(52) U.S. Cl. CPC A61K 9/06; A61K 31/131; A61K 31/167; A61K 31/198; A61K 31/245; A61K 31/445; A61K 31/455; A61K 31/4745; A61K 31/475; A61K 31/704; A61K 31/7068; A61K 47/34

(57) ABSTRACT

A sustained-release gel with insoluble salt as pH adjuster, and its preparation method and application are disclosed. The sustained-release gel is obtained by dissolving polymer in an aqueous solvent, followed by blending and compounding the alkaline inclusion and the insoluble salt crystalline powder. The insoluble salt is at solid state and thus has thus a large amount as the reservoir of the pH adjuster. The preparation is injectable, can be converted into gel state after being injected into organism, and realizes the sustained release of the inclusion at the injection site. The presence of the insoluble salt reduces the solubility of the inclusion in the gel by releasing the free salt sustainably and thus adjusting the pH inside the gel in a sustained manner. The burst release of the inclusion out of the gel is significantly alleviated.

### TECHNICAL FIELD

The disclosure belongs to the technical field of pharmaceutical preparations, and particularly relates to a sustained-release gel with insoluble salt as pH adjuster, and its preparation method and application.

### BACKGROUND ART

The sustained-release gel has wide application, but also has the problem that the pH value or its change in the gel is unfavorable for the sustained release of drugs or encapsulated substances due to factors such as the polymer itself or its degradation product. As usual, a high solubility of a drug is not conducive to its sustained release out of a gel.

Taking the gellable aqueous solution of polyester-polyether block copolymer as an example, which serves as a drug delivery system by virtue of good biocompatibility, degradability and unique thermosensitive characteristic. Particularly useful is a triblock copolymer of PLGA-PEG-PLGA consisting of polyethylene glycol (PEG) and poly(lactic-glycolic acid) (PLGA). The polymer can be synthesized through ring-opening polymerization of lactide and glycolide taking PEG as an initiator and stannous octoate as a catalyst. The triblock copolymer with polyester blocks at two ends and the PEG block in the middle can be obtained by one-step polymerization. Under the appropriate block proportion, composition and concentration, the polymer solution is in a flowable sol state at room temperature and is easy to blend with the drug; and at body temperature, it can spontaneously transform into a gel state to achieve a sustained release of the loaded drug at the injection site. In the field of pharmacy, druggability and injectability of an alkaline indissoluble drug can be increased by preparing a soluble salt-form drug. The polyester-polyether block copolymer aqueous solution has a natural acidic environment owing to the hydrolysis mode, and the eventual degradation products are lactic acid, glycolic acid and PEG. At room temperature, the pH of such a polymer aqueous solution is usually around 3, and the acidic environment facilitates the dissolution of indissoluble alkaline drugs. However, the increased solubility of a small molecular drug leads to its burst release out of a hydrogel, which is not beneficial for the long-term efficacy of the drug. Therefore, it is of great significance to solve the problem of significant burst release of a drug and realize long-acting sustained release of the drug out of the gel.

### SUMMARY

In view of the above, the first purpose of the present disclosure is to provide a sustained-release gel with insoluble salt as pH adjuster, which can significantly improve the sustained-release effect of a loaded drug or a loaded inclusion by utilizing the interaction between a dissolution product of the insoluble salt and an alkaline inclusion or a polymer and its degradation products.

In order to achieve the purpose, the disclosure adopts the following technical scheme:

A sustained-release gel with insoluble salt as pH adjuster comprises insoluble salt including its solid state, alkaline inclusion, and polymer aqueous system.

It is worth to be noted that there is an interaction between the dissolution products of the insoluble salts disclosed herein and the alkaline inclusions or polymers and their degradation products, and that the interaction is adsorption, electrostatic interaction or coordination.

The prior art mostly adopts soluble ionic compounds (such as soluble phosphate buffer solution) to adjust the pH of the system, so that the pH adjustment effect is only effective in the early stage. The disclosure provides a sustained-release gel with insoluble salt as a pH adjuster, where the insoluble salt is at solid state and thus has a large amount as the reservoir of the pH adjuster. The preparation has injectable property, can be converted into gel state after being injected into an organism, and realizes sustained-release of the inclusion at the injection site. The presence of the insoluble salt reduces the solubility of the inclusion in the gel by releasing the free salt sustainably and thus adjusting the pH inside the gel in a sustained manner. A part or all of the inclusion is dispersed in the gel network in the form of crystals, and the burst release of the inclusion from the gel is significantly alleviated in the early stage. Meanwhile, the insoluble salt at the solid state is gradually dissolved along with the generation of acidic degradation products of the biodegradable polymer constituting the skeleton of the gel in the later stage, strengthening the sustained-release of the inclusion out of the gel for a long time.

Further considering that the insoluble salt is slightly soluble or insoluble in water and can be dissolved in an acid environment, the insoluble salt includes one or more compounds of carbonate other than potassium, sodium and ammonium, and of phosphate, hydrogen phosphate, silicate or calcium sulfate, silver sulfate, magnesium ammonium phosphate and sodium bismuthate.

It is worth to be noted that there are various insoluble salts. A typical one is calcium carbonate, a mineral crystal. It can not only serve as an effective active substance for calcium supplement, but also be added into a pharmaceutical preparation as a pharmaceutical adjuvant. The calcium carbonate has the characteristics of low price, good biocompatibility, degradability, acid responsiveness and the like, and can be widely used as a drug carrier in a drug delivery system. Notably, the U.S. Food and Drug Administration (FDA) has approved calcium carbonate for use in food and pharmaceutical formulations. It is possible to entrap enzymes, DNA or drugs in calcium carbonate for the purpose of achieving delivery and release of the entrapped substances. The conventional method for entrapping the drugs into calcium carbonate includes adsorption, osmosis, and coprecipitation. In particular, due to its unique pH sensitivity, it is often used for targeted delivery of antitumor drugs, i.e., by responding to the tumor microenvironment of a partial acid, thereby achieving targeted delivery and release of the loaded antitumor drugs.

The disclosure takes advantage of the pH responsiveness of calcium carbonate and the like as a pH adjuster. The calcium carbonate and the like at the solid state as well as the drug powder are directly added into an aqueous solution of polyester-polyether block copolymer. The introduction of calcium carbonate and the like does not influence the thermal gelation behavior of the polymer solution, namely the prepared preparation is in a sol state at room temperature and thus exhibits injectability. The preparation can be converted into a physical gel state after being injected into an organism, so that the sustained-release of the drugs is realized at the injection site. The addition of calcium carbonate and the like reduces the solubility of the drug in the polymer solution by adjusting the pH inside the gel, so that a part of the drug is dispersed in the gel network in the form of crystals to guarantee the sustained drug release, while the dissolved part of the drug can, albeit minor, meet the drug efficacy. It is important that the burst release of the drug can be reduced because most of the added drug are initially at solid state. In the later stage, acidic degradation products are generated owing to the degradation of the biodegradable polymer constituting the skeleton of the gel, calcium carbonate and the like are gradually dissolved, the solubility of the alkaline drug is gradually increased, leading to the sustained release of the drug along with the gel degradation.

Further, the alkaline inclusion includes one or more of procaine, tetracaine, proparacaine, oxybuprocaine, lidocaine, bupivacaine, mepivacaine, ropivacaine, imiquimod, nitrogen mustard, gemcitabine, vinblastine, vincristine, doxorubicin, daunorubicin, irinotecan, 10-hydroxycamptothecin, nicotinamide, and arginine.

It is worth to be noted that the alkaline inclusion is an inclusion with low solubility in a normal aqueous solution and high solubility in acidic environment, contains protonatable groups. It is conventionally used after being prepared into soluble salts by adding acid clinically or pharmaceutically. But in this invention, the drug can, in the powder form, be added into the gellable system.

Further, the polymer is a polyester-polyether block copolymer, a polymer having carboxylate, sulfate, sulfonate or phosphate groups in side chains, or a mixture thereof.

It is worth to be noted that the polymer can achieve gelation in vivo, and the aqueous polymer solution or the aqueous solution after degradation thereof is acidic.

Still further, the polyester-polyether block copolymer is one or more of a triblock copolymer of ABA or BAB type, a diblock copolymer of AB type, a graft copolymer of A-g-B or B-g-A type, (AB), copolymer, A (BA), or B (AB), copolymer, wherein A is poly(ethylene glycol), and B is aliphatic polyester.

Still further, the polyether block A is poly(ethylene glycol) with molecular weight of 400-6000, and the polyester block B is aliphatic polyester with molecular weight of 500-5000.

Still further, the polyester block B is a combination of one or more of any forms of poly(D, L-lactide), poly(L-lactide), polyglycolide, poly(ε-caprolactone), poly(δ-valerolactone) and polycarbonate.

Further, the polymer having carboxylate, sulfate, sulfonate or phosphate groups in side chains is a macromolecule containing one or more blocks of acrylic acid, tert-butyl acrylate-co-acrylic acid, sodium 4-styrenesulfonate, 2-propanesulfonic acid methacrylate, methacrylic acid β-ethyl phosphate, methacrylic acid β-ethyl sulfate, glutamic acid, and aspartic acid.

Further, the preparation can be injected at room temperature, can spontaneously form physical hydrogel at body temperature, can delay the release of the carried inclusion, and an addition of the insoluble salt reduces the solubility of the inclusion in a polymer solution by adjusting the pH inside the gel so that a part or all of the inclusion is dispersed in a gel network in a crystal form. The pH value of the gel preparation for realizing sustained-release of the inclusion is 5-8.

It is worth to be noted that, different from other technologies which rely on the form of the insoluble salt of the drug to achieve the sustained release of the drug, the disclosure adjusts the pH value of the sustained/controlled release system by adding the insoluble salt, thereby reducing the solubility of the drug and enabling the drug to exist in the form of crystal, thereby realizing the sustained release of the drug. And different from other pH adjusters which are only effective in the initial stage, the insoluble salt can be dispersed in the system at solid state to continuously react with gel degradation products and the like, so that the continuous pH adjustment effect is exerted, and the sustained release of the drug is achieved along with the gel degradation.

The second purpose of the disclosure is to provide a preparation method for the sustained-release gel with insoluble salt as the pH adjuster as described above.

In order to achieve the purpose, the disclosure adopts the following technical scheme:

A preparation method for the sustained-release gel with insoluble salt as the pH adjuster includes such steps as preparing a polymer aqueous solution, and then the polymer aqueous solution is blended and compounded with the alkaline inclusion and the insoluble salt including its solid state.

Further, the preparation method includes the following specific steps:
(1) Putting one or more copolymers into an aqueous solution together, and magnetically stirring at −10-40° C. to completely dissolve the polymers to obtain a polymer aqueous system for later use;
(2) Adding alkaline inclusion and insoluble salt crystalline powders, and magnetically stirring at −10-40° C. to obtain the sustained-release gel with insoluble salt including its solid state as pH adjuster.

The third purpose of the present disclosure is to provide the application of the sustained-release gel as described above. The gel formulation releases analgesic drugs, anti-tumor drugs or medical cosmetic active agents.

In order to achieve the purpose, the disclosure adopts the following technical scheme:

The sustained-release gel with insoluble salt as pH adjuster is used as a formulation to release drugs in a sustained manner.

It is worth to be noted that, the sustained-release gel provided in the disclosure is in a flowing injectable state at room temperature and can be converted into a gel state after being injected into a body. The insoluble salt contained in the preparation reduces the solubility of the inclusion in a polymer solution by adjusting the pH inside the gel, so that a part or all of the inclusion is dispersed in a gel network in a crystal form, the release of the carried inclusion is delayed, the administration frequency is reduced, and the compliance of a patient is improved.

Further, the preparation disclosed herein may be administered in any suitable form including, without limitation, local injection such as subcutaneous injection, intramuscular injection, perineural injection, intra-articular injection, intra-tissue injection, peritumoral injection, and the like.

In some embodiments, pain, as an important vital sign, often occurs after clinical surgery. The application of local anesthetics around peripheral nerves not only effectively controls pain, but also reduces the use of opioids, which has attracted wide attention. The forms of hydrochloride, sulfonate and the like of these drugs are commonly used clinically to improve the solubility of the drug and enable the drug to be prepared into an injectable solution to exert the effect to control pain. However, these drugs have low molecular weight and short half-life, and their efficacy can only be maintained for a few hours in vivo, which is far from enough to maintain a long-term analgesic effect. It is inconvenient to use drugs frequently in clinical use. Therefore, it is of great significance to develop a long-acting preparation which is safe, convenient to use, and able to exert analgesic effect.

For example, bupivacaine is an amide local anesthetic widely used clinically, and the drug has a tertiary amine group in a structural formula and is easy to dissolve in an acidic medium. If bupivacaine and calcium carbonate crystalline powder are added into a polymer solution and the polymer solution can undergo sol-gel transition upon heating with the transition temperature between room temperature and body temperature, the bupivacaine can be spontaneously localized around nerves after the polymer sol is injected near the nerve. By this way, the problem that the solution is easy to leak through muscle gaps is solved, the pH inside the gel can be adjusted by adding the calcium carbonate, the solubility of the bupivacaine in the gel is reduced owing to the controlled intra-gel pH, and a part of the bupivacaine is dispersed in the gel network in a crystal form for a long time. As such, the burst release of bupivacaine from the gel is significantly alleviated in the early stage, and calcium carbonate is gradually dissolved along with generation of acidic degradation products of the biodegradable polymer gel in the later stage. Hence, sustained release of bupivacaine is realized, and a long-acting analgesic effect is achieved in vivo.

Based on the same inventive concept, in other embodiments, after calcium carbonate is introduced into the polymer solution, the release of an immune adjuvant imiquimod (R837) can be delayed, the effect of promoting dendritic cell maturation is continuously exerted, the cell-mediated immune response is further improved, and a better anti-tumor curative effect is obtained.

Compared with the conventional technical strategy that soluble ionic compounds such as soluble phosphate buffer solution are mostly adopted to adjust the pH of the system so that the significant pH adjusting works only in the initial stage, the disclosure distinguishes itself as employing the insoluble salt at solid state as the reservoir of the pH adjuster, and provides the sustained-release gel, its preparation method and its application. The method is facile, and the resultant preparation or formulation has injectability, can be converted into gel state after being injected into organism, and realizes sustained release of the inclusion at injection site. The insoluble salt reduces the solubility of the inclusion in the gel by adjusting the pH inside the gel, so that a part of the inclusion is dispersed in the gel network in the form of crystals, which affords a drug reservoir to be released in a sustained manner for a long time. The burst release of the inclusion out of the gel is significantly alleviated in the early stage, and the insoluble salt is gradually dissolved along with the generation of acidic degradation products of the biodegradable polymer gel in the later stage, so that the sustained release of drugs is realized. The technology can be applied to the fields of long-acting analgesia, medical cosmetology, anti-tumor therapy and the like.

### DESCRIPTION OF THE ACCOMPANYING DRAWINGS

In order to more clearly illustrate the embodiments of the present disclosure or the technical solutions in the prior art, the drawings used in the description of the embodiments or the prior art will be briefly described below. The drawings in the following description are only embodiments of the disclosure and other drawings may be derived from the provided drawings by those skilled in the art without inventive effort.

FIG. 1 presents the global view of the sustained-release preparation P1 in embodiment 1 of the present disclosure at room temperature and body temperature.

FIG. 2 shows an in-vivo result to demonstrate a gel in-situ formed after injecting preparation P1 around the sciatic nerve of a rat in embodiment 2 of the present disclosure.

FIG. 3 displays the in-vitro release curve of the thermosensitive gel encapsulating ropivacaine (ROP) in each preparation group in embodiment 3 of the present disclosure.

FIG. 4 is a histograph showing the pH of each preparation group in embodiment 4 of the present disclosure.

FIG. 5 presents the in vivo data to evaluate the analgesic effect in each preparation group in embodiment 5 of the present disclosure.

FIG. 6 shows an in-vitro release curve of each group of preparation R837 in embodiment 6 of the present disclosure.

### DETAILED DESCRIPTION OF THE EMBODIMENTS

The technical solutions in the embodiments of the present disclosure will be clearly and completely described below. The described embodiments are only some embodiments of the disclosure, not all embodiments. All other embodiments, which can be obtained by a person skilled in the art without making any creative effort based on the embodiments in the present disclosure, belong to the protection scope of the present disclosure.

The term “embodiment” dedicated herein is not construed as superior or better than other embodiments as any embodiment described as “exemplary”. Performance index tests in the embodiments of this application, unless otherwise indicated, were performed using routine experimentation in the art. It is to be understood that the terminology used herein is for the purpose of describing particular embodiments only and is not intended to be limiting of the disclosure.

Unless defined otherwise, technical and scientific terms used herein have the same meaning as commonly understood by one of ordinary skill in the art to which this application belongs; other test methods and techniques not specifically mentioned herein are those commonly employed by those of ordinary skill in the art.

The terms “substantially” and “about” are used herein to describe small fluctuations. For example, they may mean less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Numerical data represented or presented herein in a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. For example, a numerical range of “1% to 5%” should be interpreted to include not only the explicitly recited values of 1% to 5%, but also include individual values and sub-ranges within the indicated range. Thus, included in this numerical range are individual values, such as 2%, 3.5%, and 4%, and sub-ranges, such as 1% to 3%, 2% to 4%, and 3% to 5%, etc. This principle applies equally to ranges reciting only one numerical value. Moreover, such an interpretation applies regardless of the breadth of the range or the characteristics being described.

In the following detailed description, numerous specific details are set forth in order to provide a better understanding of the present disclosure. It will be understood by those skilled in the art that the present application may be practiced without some of these specific details. In the embodiments, some methods, means, instruments, apparatuses, etc. known to those skilled in the art are not described in detail in order to highlight the subject matter of the present application.

On the premise of no conflict, the technical features disclosed in the embodiments of the present application may be combined arbitrarily, and the obtained technical solution belongs to the content disclosed in the embodiments of the present application.

### Embodiment 1

A Sustained-Release Gel with Insoluble Salt as pH Adjuster, its Preparation Method and Application:

A thermoinduced hydrogel or thermogel as sustained-release analgesic preparation P1 was prepared from polyester-polyether block copolymer, PLGA-PEG-PLGA (LA/GA=2.5/1) 2 g with a molecular weight of 1262-1500-1262, PLGA-PEG-PLGA (LA/GA=3/1) 2 g with a molecular weight of 1429-1000-1429, ropivacaine 0.45 g, and calcium carbonate 0.02 g.

Step (1): putting the block copolymers with two molecular weights into water, and magnetically stirring for 24 hours at 4° C. to prepare a polymer micelle solution with the polymer concentration of 26 wt %;

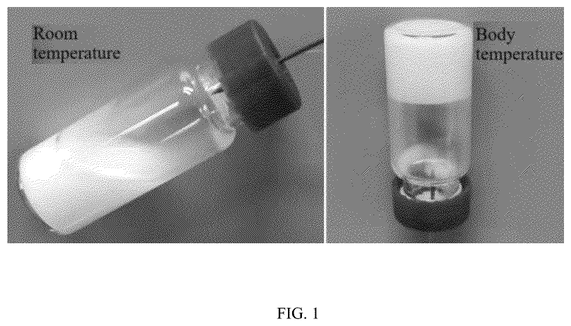

Step (2): adding 0.45 g of ropivacaine and 0.02 g of calcium carbonate into the polymer micelle solution prepared in step (1), adding an appropriate amount of water to dilute to a formulation prescription of 25 wt % of the polymer, 4.5 wt % of ropivacaine and 0.2 wt % of calcium carbonate, and stirring at 4° C. for 12 h to form an injectable suspension. The sol-gel transition upon heating of such an thermogellable system is demonstrated in FIG. 1, where the solid-state insoluble salt powder and a part of insoluble ropivacaine made the aqueous system white. The preparation can be injected around sciatic nerve of rat to form in-situ gel around sciatic nerve for long-acting analgesia.

In the resulting preparation, calcium ions generated by partial dissolution of CaCO₃ can coordinate with oxygen lone pair electrons in the PEG block in the block copolymer PLGA-PEG-PLGA, and can also coordinate with carbonyl oxygen lone pair electrons in the ropivacaine structure; the undissolved CaCO₃ can absorb the ropivacaine in the preparation by adsorption.

### Embodiment 2

An Application of the Sustained-Release Gel with Insoluble Salt as pH Adjuster:

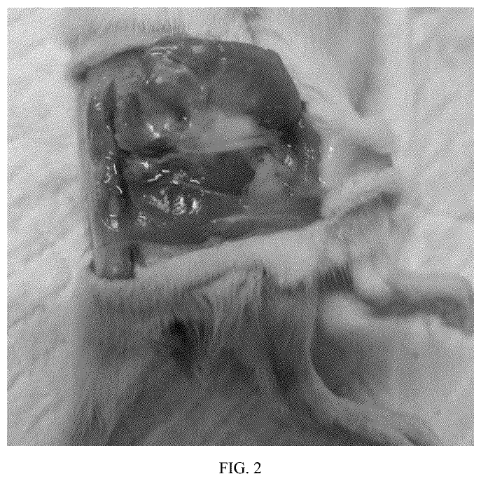

The isoflurane anesthetized rat was laid on their sides with the femurs kept perpendicular to the trunk. The greater trochanter and ischial tuberosities were found by palpation, the pharmaceutical preparation disclosed in embodiment 1 was inserted through the syringe needle from the posterior medial side of the greater trochanter, advanced antero-medially, and after contacting the ischial surface, the needle was withdrawn by about 1 mm, after which the preparation disclosed in embodiment 1 was pushed around the sciatic nerve of the rat. After several hours, the rat was dissected and observed for in-situ gel formation around the sciatic nerve, as shown in FIG. 2.

### Embodiment 3

The Performance Research of the Sustained-Release Gel with Insoluble Salt as pH Adjuster:

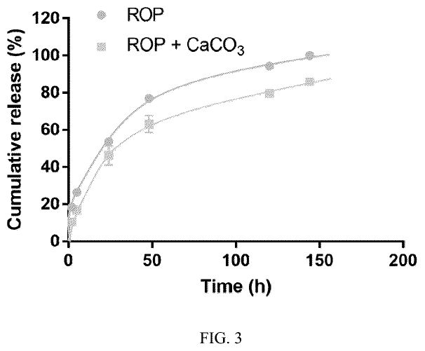

The preparation P1 disclosed in embodiment 1 and its control preparation C1 (25 wt % polymer, 4.5 wt % ropivacaine) were placed in a dialysis card with a molecular weight cut-off of 3500 to a pre-gel. A quantity of release fluid was withdrawn at specific time points, an in-vitro release profile was calculated and plotted by measuring the drug concentration in the release fluid at each sampling time point. The addition of calcium carbonate delayed the release of ropivacaine from the gel, as shown in FIG. 3.

### Embodiment 4

The Performance Research of the Sustained-Release Gel with Insoluble Salt as pH Adjuster:

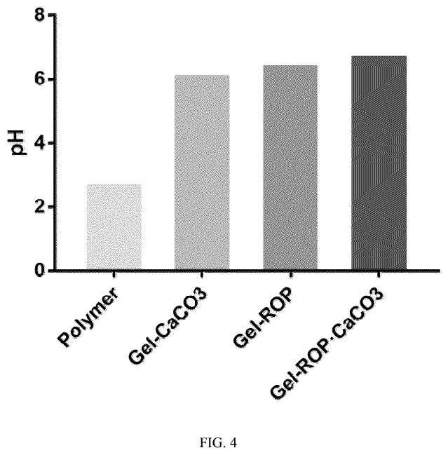

The pH values of the polymer solution (25 wt % polymer), preparation P1 disclosed in embodiment 1 and its control preparation C1 (25 wt % polymer, 4.5 wt % ropivacaine), C2 (25 wt % polymer, 0.2 wt % calcium carbonate) were measured with pH meters, as shown in FIG. 4.

### Embodiment 5

The Performance Research of the Sustained-Release Gel with Insoluble Salt as pH Adjuster:

SD male rats of 20, about 350 g were selected and randomly divided into four groups. Group A is the polymer solution (25 wt % polymer), group B is the bupivacaine (BUP) hydrochloride solution (0.5 wt %), group C is the bupivacaine-including polymer gel (25 wt % polymer, 8 wt % bupivacaine), and group D is bupivacaine-including and calcium-carbonate-including polymer gel (25 wt % polymer, 8 wt % bupivacaine, 0.2 wt % calcium carbonate). In each group, 0.5 mL of the corresponding sol was injected around sciatic nerves of the rats.

The in-vivo analgesic effect was evaluated by hot plate method. The hot plate temperature was set at 56° C., and the time required for the rat to lift the hind paw was taken as the thermal latency value. The basic thermal latency of each rat was examined first. Each rat was measured three times at the same time point, and the average value was taken as the basic thermal latency. The maximum allowable thermal latency was controlled to be 12 s. If a rat did not have any response after 12 s, the foot of the rat was lifted off the hot plate to avoid the tissue damage of the foot of the rat. Each preparation group was injected around the sciatic nerves of rats. Each rat was tested three times at each time point, and the consecutive two measurements were separated by 30 s. Each measurement was obtained as a real-time thermal latency value. The result of thermal latency is expressed as maximum proportionality effect (MPE) and is calculated as follows:

$$MPE ( \% ) = \frac{C - B}{P - B} \times 100 \%$$

Here, B is the basal thermal latency of the rat, P is the maximum allowable thermal latency, and C is the real-time thermal latency of the rat at each test time point. The effective sensory retention time is defined as the time that lasts from injection of the preparation to a recovery of 50% of the MPE value.

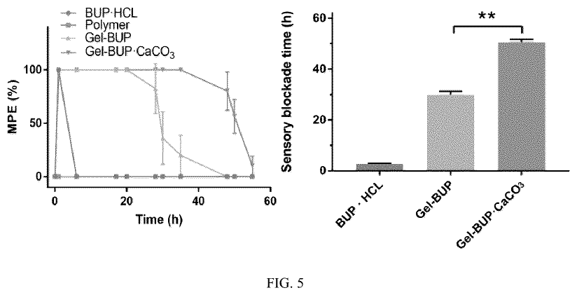

The effective sensory retention duration for each preparation group resulted as follows: group B 2.71±0.26 h, group C 29.91±1.41 h, and group D 50.63±1.08 h, as shown in FIG. 5.

### Embodiment 6

A Sustained-Release Gel with Insoluble Salt as pH Adjuster, its Preparation Method and Application:

The thermogel sustained-release anti-tumor preparation P2 was prepared from 1 g of PLGA-PEG-PLGA (LA/GA 4/1) with a molecular weight of 1250-1500-1250, 1 g of PLGA-PEG-PLGA (LA/GA 4/1) with a molecular weight of 1500-1000-150, and 5 mg of R837; the thermogel sustained-release anti-tumor preparation P3 was prepared from 1 g of PLGA-PEG-PLGA (LA/GA 4/1) with a molecular weight of 1250-1500-1250, 1 g of PLGA-PEG-PLGA (LA/GA 4/1) with a molecular weight of 1500-1000-1500, 5 mg of R837 and 5 mg of calcium carbonate; the thermogel sustained-release anti-tumor preparation P4 was prepared from 1 g of PLGA-PEG-PLGA (LA/GA 4/1) with a molecular weight of 1250-1500-1250, 1 g of PLGA-PEG-PLGA (LA/GA 4/1) with a molecular weight of 1500-1000-1500, 5 mg of R837 and 15 mg of calcium carbonate.

Step (1): putting the block copolymers with two molecular weights into water, and magnetically stirring for 24 hours at 4° C. to prepare a polymer micelle solution with the polymer concentration of 20 wt %;

Step (2): adding the above amounts of R837 and calcium carbonate to the polymer micelle solution prepared in step (1), and stirring at 4° C. for 12 h to form an injectable suspension. The preparation can be injected around a mouse tumor to form an in-situ gel around the tumor, so as to achieve long-acting anti-tumor effects.

In the resulting preparation, calcium generated by partial dissolution of CaCO₃ can coordinate with oxygen lone pair electrons in the PEG block in PLGA-PEG-PLGA; the undissolved CaCO₃ can absorb the R387 in the preparation by adsorption.

### Embodiment 7

The Performance Research of the Sustained-Release Gel with Insoluble Salt as pH Adjuster:

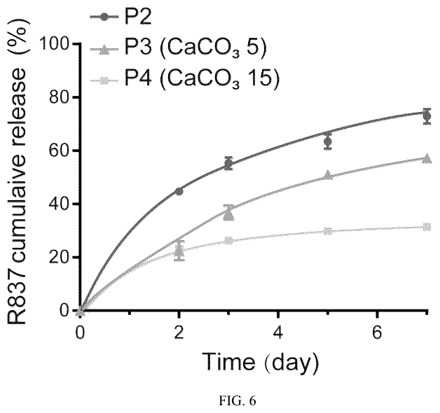

The compound preparations P2, P3 and P4 were prepared according to the method described in embodiment 6. 10 mL cylindrical glass bottle was placed in a water bath shaker at 37° C., 0.5 g of P2, P3, and P4 preparations were added to the bottom of the glass bottle separately and gelled at a constant temperature for 10 min, and 50 mL of PBS buffer was added to each glass bottle. At a fixed time point, 5 mL of the liquid in the cylindrical glass bottle was extracted, an equal amount of fresh deionized water was replaced, the concentration of R837 in the extracted sample was measured by an ultraviolet spectrophotometer, converted into an accumulated release rate of the drug. The measured accumulated release curve is shown in FIG. 6.

### Embodiment 8

A Sustained-Release Gel with Insoluble Salt as pH Adjuster, its Preparation Method and Application:

The gel sustained-release anti-tumor preparation P5, wherein the polyester-polyether block copolymer was mPEG-PLGA diblock copolymer, the molecular weight was 600-1000, the polymer mass was 2 g, irinotecan was 0.05 g, and calcium sulfate was 0.05 g.

Step (1): putting 2 g of the block copolymers in physiological saline, and magnetically stirring for 24 hours at 4° C. to prepare a polymer micelle solution with the polymer concentration of 25 wt %;

step (2): adding 0.05 g of irinotecan and 0.05 g of calcium sulfate into the polymer micelle solution prepared in the step (1), adding a proper amount of water to dilute the solution until the preparation formula was 20 wt % of polymer, 0.5 wt % of irinotecan and 0.5 wt % of calcium sulfate, and stirring the mixture at 25° C. for 12 h to form the injectable sustained-release preparation. The preparation was injected around a mouse tumor to form an in-situ gel around the tumor, so as to achieve long-acting anti-tumor effects.

In the resulting preparation, calcium generated by partial dissolution of CaSO₄ can coordinate with oxygen lone pair electrons in the PEG block in mPEG-PLGA, and can also coordinate with carbonyl oxygen lone pair electrons in the irinotecan structure; the undissolved CaSO₄ can absorb the irinotecan in the preparation by adsorption.

### Embodiment 9

A Sustained-Release Gel with Insoluble Salt as pH Adjuster, its Preparation Method and Application:

The gel filling anti-aging preparation P6, wherein the polyester-polyether block copolymer was PLGA-PEG-PLGA triblock copolymer, the molecular weight was 1850-1500-1850 (LA/GA 4/1), the polymer mass was 2 g, arginine is 0.03 g, and calcium hydrogen phosphate was 0.02 g.

Step (1): putting 2 g of the triblock copolymer into deionized water, and magnetically stirring at 40° C. until the polymer was completely dissolved to prepare a polymer micelle solution with the polymer concentration of 20 wt %;

step (2): adding 0.03 g of arginine and 0.02 g of calcium hydrogen phosphate into the polymer micelle solution prepared in the step (1), adding a proper amount of water to dilute the solution until the preparation formula is 15 wt % of polymer, 0.3 wt % of arginine and 0.2 wt % of calcium hydrogen phosphate, and placing the mixture at 5° C. to stir and mix uniformly to form the injectable sustained-release preparation. The preparation was injected into the periphery of skin aging tissue by intramuscular injection to form an in-situ gel at the part to be filled, so as to realize tissue filling and long-acting anti-aging.

In the resulting preparation, calcium generated by partial dissolution of CaHPO₄ can coordinate with oxygen lone pair electrons in the PEG block in PLGA-PEG-PLGA, and can also coordinate with carbonyl oxygen lone pair electrons in the arginine structure; the undissolved CaHPO₄ can absorb the arginine in the preparation by adsorption.

### Embodiment 10

A Sustained-Release Gel with Insoluble Salt as pH Adjuster, its Preparation Method and Application:

A gel sustained-release anti-tumor preparation P7 was prepared. Methyl cellulose (MC) and γ-poly(glutamic acid) (γ-PGA) were used as raw materials to prepare a methyl cellulose stearate/γ-PGA composite gel by means of an esterification reaction. Firstly, 0.15 g of stearic acid and a proper amount of DMF were taken, stirred and dissolved, then 0.2 g of dicyclohexylcarbodiimide (DCC) and 0.005 g of dimethylaminopyridine (DMAP) were added. After the mixture reacted for 20 min in an ice bath, 0.3 g of MC was added, and the mixture was placed at room temperature for additional reaction for 24 h. After the reaction was finished, the reaction solution was slowly added to 250 mL of absolute ethyl alcohol for precipitation, purified, filtered and dried in vacuum to obtain methyl cellulose stearate (MCS). Then 0.2 g of MCS and 0.2 g of γ-PGA were taken and put into deionized water, stirred under the ice bath condition until the system was viscous and bubbles were generated. The system was completely dissolved and the bubbles were eliminated after refrigeration. Then 0.1 g of vinblastine and 0.02 g of magnesium silicate powder were directly added into the above polymer solution, and stirred and mixed uniformly at the temperature of −10° C. to form the injectable sustained-release preparation. The preparation was injected around a mouse tumor to form an in-situ gel around the tumor to realize long-acting anti-tumor efficacy.

In the resulting preparation, magnesium generated by partial dissolution of MgSiO₃ can coordinate with carbonyl oxygen lone pair electrons in the MCS/γ-PGA, and can also coordinate with carbonyl oxygen lone pair electrons in the vinblastine structure; the undissolved MgSiO₃ can absorb the vinblastine in the preparation by adsorption.

### Embodiment 11

A Sustained-Release Gel with Insoluble Salt as pH Adjuster, its Preparation Method and Application:

A gel sustained-release anti-tumor preparation P8 was prepared. A poly(aspartic acid) cross-linked poly(N-isopropylacrylamide/acrylic acid) (P(NIPAAm/AAc)) thermosensitive hydrogel was synthesized by using N-acryloyl chloroethyl polyaspartic imide (NAE-PAI) as a cross-linking agent. Firstly, NIPAAm, AAc (NIPAAm/AAc 97.5/2.5) and the cross-linking agent NAE-PAI (0.5%) were added to 50 mL of phosphate buffer solution to completely dissolve the monomers. The nitrogen was introduced to remove dissolved oxygen in the solution, and ammonium persulfate and N, N, N, N-tetramethyl ethylenediamine were added as an initiator and an accelerator, respectively. After vigorously stirring for 1 h, the nitrogen introduction was stopped, and the reaction was carried out at room temperature for 24 h. After the reaction was completed, the unreacted monomers were removed by washing with distilled water. The polymer was obtained after freeze drying. Then, 2 g of the polymer was taken and dissolved in water to formulate a polymer solution, and 0.3 g of doxorubicin and 0.03 g of barium phosphate powder were added into the polymer solution. The solution was placed at 40° C., stirred and mixed until uniform, so as to form an injectable sustained-release preparation. The preparation was injected around a mouse tumor to form an in-situ gel around the tumor to realize long-acting anti-tumor efficacy.

In the resulting preparation, barium generated by partial dissolution of Ba₃(PO₄)₂ can coordinate with carbonyl oxygen lone pair electrons in the poly(aspartic acid) cross-linked poly(N-isopropylacrylamide/acrylic acid), and can also coordinate with carbonyl oxygen lone pair electrons in the doxorubicin structure; the undissolved Ba₃(PO₄)₂ can absorb the doxorubicin in the preparation by adsorption.

### Embodiment 12

A Sustained-Release Gel with Insoluble Salt as pH Adjuster, its Preparation Method and Application:

A gel sustained-release analgesic preparation P9 was prepared. First, 2.28 g of N-isopropylacrylamide was taken, an appropriate amount of acetone and distilled water were added, and the N-isopropylacrylamide was completely dissolved by stirring. After freezing with liquid nitrogen and vacuumizing, a high-purity argon gas was introduced, 20 mg of cuprous chloride and tris(2-methylaminoethyl)amine were added. After freezing-evacuating-argon circulation, 0.1 mmol of bis [2-(2-bromo-isobutyryloxy)ethyl]disulfide was added, and poly(N-isopropylacrylamide) was prepared by reaction at 0° C. for 12 h. Then, 0.02 mol of oxygen-free tert-butyl acrylate was added into the system, reacted for 24 h. The product was transferred out, filtered by suction, dialyzed and freeze-dried to obtain poly(tert-butyl acrylate)-b-poly(N-isopropylacrylamide)-b-poly(tert-butyl acrylate). The product was subjected to ether precipitation-chloroform redissolution, and then dried in vacuum to obtain a poly(tert-butyl acrylate)-b-poly(N-isopropylacrylamide)-b-poly(tert-butyl acrylate)triblock polymer. Then, 2 g of the polymer was taken, dissolved in water, placed at 40° C. and stirred to complete dissolution; and 0.05 g of lidocaine and 0.02 g of calcium sulfate powder were added, and then placed at 15° C., stirred and mixed until uniform to form an injectable sustained-release preparation. The preparation was injected into a rat joint cavity to form an in-situ gel in the joint cavity to realize long-acting analgesia.

In the resulting preparation, calcium generated by partial dissolution of CaSO₄ can coordinate with carbonyl oxygen lone pair electrons in the poly(tert-butyl acrylate)-b-poly(N-isopropylacrylamide)-b-poly(tert-butyl acrylate), and can also coordinate with carbonyl oxygen lone pair electrons in the lidocaine structure; the undissolved CaSO₄ can absorb the lidocaine in the preparation by adsorption.

### Embodiment 13

A Sustained-Release Gel with Insoluble Salt as pH Adjuster, its Preparation Method and Application:

A thermogel sustained-release analgesic preparation P10 was prepared. First, 2.28 g of N-isopropylacrylamide was taken, an appropriate amount of acetone and distilled water were added, and the N-isopropylacrylamide was completely dissolved by stirring. After freezing with liquid nitrogen and vacuumizing, a high-purity argon gas was introduced, 20 mg of cuprous chloride and tris(2-methylaminoethyl)amine were added. After freezing-evacuating-argon circulation, 0.1 mmol of bis [2-(2-bromo-isobutyryloxy)ethyl]disulfide was added, and poly(N-isopropylacrylamide) was prepared by reaction at 0° C. for 12 h. 0.3 g of poly(N-isopropylacrylamide) was dissolved in an appropriate amount of methylpyrrolidone, and a dimethyl sulfoxide solution of 0.3 mmol/mL of methacrylic acid-2-propanesulfonic acid was added under the protection of argon, then cuprous bromide and 1,1,4,7,10,10-hexamethyltriethylenetetramine were added and reacted at 80° C. for 10 h. The product was separated and purified, and dried in vacuum to obtain a poly(methacrylic acid)-2-propane sulfonic acid-poly-(N-isopropylacrylamide)-b-poly(methacrylic acid)-2-propane sulfonic acid triblock polymer. Then, 1 g of the polymer was taken, dissolved in water, placed at 25° C. and stirred to complete dissolution; 0.02 g of tetracaine and 0.015 g of aluminum phosphate (AlPO₄) powder were added. It was then placed at 30° C. and stirred for 12 h, forming an injectable sustained-release preparation. The preparation was injected into a rat joint cavity to form an in-situ gel in the joint cavity, so as to realize long-acting analgesia.

In the resulting preparation, aluminum generated by partial dissolution of AlPO₄ can coordinate with carbonyl oxygen lone pair electrons in the poly(methacrylic acid)-2-propane sulfonic acid-poly-(N-isopropylacrylamide)-b-poly(methacrylic acid)-2-propane sulfonic acid, and can also coordinate with carbonyl oxygen lone pair electrons in the tetracaine structure; the undissolved AlPO₄ can absorb the tetracaine in the preparation by adsorption.

### Embodiment 14

A Sustained-Release Gel with Insoluble Salt as pH Adjuster, its Preparation Method and Application:

A gel sustained-release anti-tumor preparation P11 was prepared. Using ethyl 2-bromopropionate as macromolecular initiator, triethylene glycol ether methacrylate, cuprous bromide and 2-2′-bipyridine were reacted at 60° C. for 5 h, and diluted by tetrahydrofuran. The catalyst cuprous bromide was removed by alumina column. The product was dried in vacuum to obtain poly(oligo ethylene glycol methyl ether methacrylate). Then, 0.3 g of poly(oligo ethylene glycol ether methacrylate) was dissolved in an appropriate amount of methylpyrrolidone, and a dimethyl sulfoxide solution of 0.3 mmol/mL of sodium 4-styrene sulfonate was added under the protection of argon. Then, cuprous bromide and 1,1,4,7,10,10-hexamethyltriethylenetetramine were added and reacted at 120° C. for 24 h. After post-treatment, poly(sodium 4-styrene sulfonate)-b-poly(oligo ethylene glycol methyl ether methacrylate) diblock polymer was obtained. 1.5 g of the polymer was taken, dissolved in water, placed at −2° C. and stirred to complete dissolution. Then, 0.015 g of 10-hydroxycamptothecin and 0.01 g of iron phosphate powder were added, placed at 0° C. and stirred for 12 h to form an injectable sustained-release preparation. The preparation was injected around a mouse tumor to form an in-situ gel around the tumor to realize long-acting anti-tumor efficacy.

In the resulting preparation, iron ions generated by partial dissolution of FePO₄ can coordinate with the oxygen lone pair electrons of the ethylene glycol units in the poly(sodium 4-styrene sulfonate)-b-poly(oligo ethylene glycol methyl ether methacrylate), and can also coordinate with carbonyl oxygen lone pair electrons in 10-hydroxycamptothecin; the undissolved FePO₄ can absorb the tetracaine in the preparation by adsorption.

### Embodiment 15

A Sustained-Release Gel with Insoluble Salt as pH Adjuster, its Preparation Method and Application:

A gel sustained-release anti-tumor preparation P11 was prepared. First, 1.5 g PCLA-PEG-PCLA with molecular weight of 1800-1500-1800 was dissolved in water and magnetically stirred at −5° C. for 48 h to form a polymer solution. Then, 0.2 g 10-hydroxycamptothecin and 0.01 g zinc silicate powder were added and mixed evenly at −5° C. to form an injectable sustained-release preparation. The preparation was injected around a mouse tumor to form an in-situ gel around the tumor to realize long-acting anti-tumor efficacy.

In the resulting preparation, zinc ions generated by partial dissolution of Zn₂SiO₄ can coordinate with the oxygen lone pair electrons of the PEG block in PCLA-PEG-PCLA, and can also coordinate with carbonyl oxygen lone pair electrons in 10-hydroxycamptothecin; the undissolved Zn₂SiO₄ can absorb the tetracaine in the preparation by adsorption.

### Embodiment 16

A Sustained-Release Gel with Insoluble Salt as pH Adjuster, its Preparation Method and Application:

A gel sustained-release analgesic preparation P13 was prepared from 2 g of PEG-PLGA-PEG with molecular weight of 550-2780-550, 0.5 g of oxybuprocaine and 0.1 g of aluminum carbonate.

Step (1): putting 2 g of triblock copolymer into water, and magnetically stirring at −10° C. to prepare a polymer micelle solution with the polymer concentration of 20 wt %;

Step (2): adding 0.5 g of oxybuprocaine and 0.02 g of Al₂(CO₃)₃ into the polymer micelle solution prepared in step (1), adding an appropriate amount of water to dilute to a formulation prescription of 15 wt % of the polymer, 5 wt % of oxybuprocaine and 1 wt % of Al₂(CO₃)₃, and stirring at 30° C. for 12 h to form an injectable suspension. The preparation was injected around sciatic nerve of a rat to form in-situ gelling around sciatic nerve for long-acting analgesia.

In the resulting preparation, aluminum ions generated by the partial dissolution of Al₂(CO₃)₃ can coordinate with the oxygen lone pair electrons of the PEG block in PCLA-PEG-PCLA, and can also coordinate with carbonyl oxygen lone pair electrons in the oxybuprocaine structure; the undissolved Al₂(CO₃)₃ can absorb the oxybuprocaine in the preparation by adsorption.

### Embodiment 17

A Sustained-Release Gel with Insoluble Salt as pH Adjuster, its Preparation Method and Application:

A gel sustained-release analgesic preparation P14 was prepared. Poly(γ-propyl-L-glutamate-grafted-oligopolyethylene glycol) (PPLGn-g-OEGm) was synthesized by multi-step reactions such as an esterification reaction, a cyclization reaction, a ring-opening polymerization, an etherification reaction, a 1,3-dipolar cycloaddition. 2 g of PPLG₈₈-g-OEG₂ with a molecular weight 32500 was dissolved in water and stirred at 30° C. to completely dissolve the polymer, and 0.01 g of propantheline and 0.01 g silver sulfate powder were added and stirred at −2° C. for 5 h to form an injectable sustained-release preparation. The preparation was injected around sciatic nerve of a rat to form an in-situ gel around sciatic nerve for long-acting analgesia.

In the resulting preparation, silver ions generated by the partial dissolution of Ag₂SO₄ can coordinate with the oxygen lone pair electrons of the ethylene glycol units in poly(γ-propyl-L-glutamate-grafted-oligopolyethylene glycol), can coordinate with carbonyl oxygen lone pair electrons in the structure, and can also coordinate with carbonyl oxygen lone pair electrons in the propantheline structure.

### Embodiment 18

A Sustained-Release Gel with Insoluble Salt as pH Adjuster, its Preparation Method and Application:

A gel sustained-release analgesic preparation P15 was prepared. First, 0.5 g of bis-amino PEG₂₀₀₀ was added into 50 ml of toluene to remove water azeotropically, and 3.99 g of γ-benzyl-L-glutamic acid-N-carboxyl cyclic internal anhydride (BGL-NCA) monomer was added into a mixed solvent of CHCl₃/DMF, and stirred and mixed at room temperature. Under the protection of argon, the mixed monomer solution was transferred to dry NH₂-PEG₂₀₀₀—NH₂. The temperature of the system was increased to 37° C. After reacting for 3 days, the system was cooled to room temperature, and 22 ml of CHCl₃ was added. After the product was completely dissolved, the mixture was slowly added dropwise to a mixed solvent of acetic acid and methanol for settling, obtaining a white solid. The triblock polymer PBGL-PEG₂₀₀₀-PBGL was obtained after vacuum drying. Then, the benzyl protecting group was removed by catalytic hydrogenation to obtain the final product PGL-PEG₂₀₀₀-PGL. After 2 g of PGL-PEG₂₀₀₀-PGL with a molecular weight of 5500 was dissolved in normal saline and stirred at 20° C. to complete dissolution, 0.01 g of procaine and 0.008 g of magnesium hydrogen phosphate powder were added and stirred at 8° C. for 12 h to form an injectable sustained-release preparation. The preparation was injected around sciatic nerves of rats to form an in-situ gel around sciatic nerve for long-acting analgesia.

In the resulting preparation, magnesium ions generated by the partial dissolution of MgHPO₄ can coordinate with the oxygen lone pair electrons of the PEG block in PGL-PEG₂₀₀₀-PGL, and can also coordinate with carbonyl oxygen lone pair electrons in the procaine structure; the undissolved MgHPO₄ can absorb the procaine in the preparation by adsorption.

### Embodiment 19

A Sustained-Release Gel with Insoluble Salt as pH Adjuster, its Preparation Method and Application:

A gel sustained-release anti-tumor preparation P16 was prepared. First, 1.5 g of poly(methacrylic acid β-ethyl phosphate)-poly(N-vinylbutylamide) was dissolved in normal saline and stirred at 2° C. to complete dissolution. Then, 0.01 g of nitrogen mustard, 0.05 g of imiquimod and 0.005 g of calcium silicate powder were added and stirred at 8° C. for 12 h to form an injectable sustained-release preparation. The preparation was injected around a mouse tumor to form an in-situ gel around the tumor to realize long-acting anti-tumor efficacy.

In the resulting preparation, calcium ions generated by the partial dissolution of CaSiO₃ can coordinate with the carbonyl oxygen lone pair electrons in poly(methacrylic acid β-ethyl sulfate)-poly(N-vinylbutylamide), and can also coordinate with oxygen lone pair electrons in the nitrogen mustard structure; the undissolved CaSiO₃ can absorb the procaine in the nitrogen mustard by adsorption.

### Embodiment 20

A Sustained-Release Gel with Insoluble Salt as pH Adjuster, its Preparation Method and Application:

A gel sustained-release anti-tumor preparation P17 was prepared. First, 1.5 g of poly(methacrylic acid β-ethyl sulfate)-poly(N-vinylpyrrolidone) was dissolved in normal saline and stirred at 12° C. to complete dissolution. Then, 0.008 g of gemcitabine, 0.005 g of magnesium ammonium phosphate powder were added and stirred at 2° C. for 12 h to form an injectable sustained-release preparation. The preparation was injected around a mouse tumor to form an in-situ gel around the tumor to realize long-acting anti-tumor efficacy.

In the resulting preparation, magnesium ions generated by the partial dissolution of MgNH₄PO₄ can coordinate with the carbonyl oxygen lone pair electrons of the poly(N-vinylpyrrolidone) block in poly(methacrylic acid β-ethyl sulfate)-poly(N-vinylpyrrolidone), and can also coordinate with carbonyl oxygen lone pair electrons in the gemcitabine structure; the undissolved MgNH₄PO₄ can absorb the procaine in the gemcitabine by adsorption.

The previous description of the disclosed embodiments is provided to enable any person skilled in the art to make or use the present disclosure. Various modifications to these embodiments is readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A sustained-release gel with insoluble salt as pH adjuster, wherein the sustained-release gel comprises an insoluble salt including its solid state, an alkaline inclusion and a polymer aqueous system.

2. The sustained-release gel with insoluble salt as pH adjuster according to claim 1, wherein the insoluble salt is slightly soluble or insoluble in water and soluble in an acidic environment, and comprises one or more compounds of carbonate other than potassium, sodium and ammonium, and of phosphate, hydrogen phosphate, silicate or calcium sulfate, silver sulfate, magnesium ammonium phosphate and sodium bismuthate.

3. The sustained-release gel with insoluble salt as pH adjuster according to claim 1, wherein the alkaline inclusion comprises one or more of procaine, tetracaine, proparacaine, oxybuprocaine, lidocaine, bupivacaine, mepivacaine, ropivacaine, imiquimod, nitrogen mustard, gemcitabine, vinblastine, vincristine, doxorubicin, daunorubicin, irinotecan, 10-hydroxycamptothecin, nicotinamide, and arginine.

4. The sustained-release gel with insoluble salt as pH adjuster according to claim 1, wherein the polymer is a polyester-polyether block copolymer, a polymer having carboxylate, sulfate, sulfonate or phosphate groups in side chains, or a mixture thereof.

5. The sustained-release gel with insoluble salt as pH adjuster according to claim 4, wherein the polyester-polyether block copolymer is one or more of a triblock copolymer of ABA or BAB type, a diblock copolymer of AB type, a graft copolymer of A-g-B or B-g-A type, (AB)ₙ copolymer, A(BA)ₙ or B(AB)ₙ copolymer, wherein A is poly(ethylene glycol), and B is aliphatic polyester.

6. The sustained-release gel with insoluble salt as pH adjuster according to claim 5, wherein the polyether block A is poly(ethylene glycol) with molecular weight of 400-6000, and the polyester block B is aliphatic polyester with molecular weight of 500-5000.

7. The sustained-release gel with insoluble salt as pH adjuster according to claim 6, wherein the polyester block B is a combination of one or more of any forms of poly(D, L-lactide), poly(L-lactide), polyglycolide, poly(ε-caprolactone), poly(δ-valerolactone) and polycarbonate.

8. The sustained-release gel with insoluble salt as pH adjuster according to claim 4, wherein the polymer having carboxylate, sulfate, sulfonate or phosphate groups in side chains is a macromolecule containing one or more blocks of acrylic acid, tert-butyl acrylate-co-acrylic acid, sodium 4-styrenesulfonate, 2-propanesulfonic acid methacrylate, methacrylic acid β-ethyl phosphate, methacrylic acid β-ethyl sulfate, glutamic acid, and aspartic acid.

9. The sustained-release gel with insoluble salt as pH adjuster according to claim 1, wherein the preparation can be injected at normal temperature, can spontaneously form a physical hydrogel at body temperature, can delay the release of the carried inclusion; an addition of the insoluble salt reduces the solubility of the inclusion in the gel by releasing the free salt sustainably and thus adjusting the pH inside the gel; a part or all of the inclusion is dispersed in the gel network in the form of crystals; the pH value of the gel preparation for realizing sustained-release of the inclusion is 5-8.

10. A preparation method for the sustained-release gel according to claim 1, wherein the polymer is prepared into an aqueous solution, and then the polymer aqueous solution is blended and compounded with the alkaline inclusion and the insoluble salt including its solid state.

11. The preparation method according to claim 10, wherein the preparation method comprises following specific steps:
(1) putting one or more polymers into an aqueous solution, and magnetically stirring at −10-40° C. to complete dissolution to obtain a polymer aqueous system for later use;
(2) adding alkaline inclusion and insoluble salt crystalline powders, and magnetically stirring at −10-40° C. to obtain the sustained-release gel with insoluble salt including its solid state as pH adjuster.

12. An application of the sustained-release gel according to claim 1, wherein the sustained-release gel is used as a formulation to release drugs in a sustained manner.

13. The application of the sustained-release gel according to claim 12, wherein the gel formulation releases analgesic drugs, anti-tumor drugs or medical cosmetic active agents.
